(19) United States
(12) Patent Application Publication
(10) Pub. No.: US 2009/0030058 A1
(43) Pub. Date: Jan. 29, 2009

(54) TOSYLATE SALT OF 6- (4-BR0M0-2-CHL0R0PHENYLAMIN0) -7-FLUORO-N- (2-HYDROXYETHOXY) -3-METHYL-3H-BENZIMI DAZOLE- 5 - CARBOXAMIDE , MEK INHIBITOR USEFUL IN THE TREATMENT OF CANCER

(76) Inventors: Mohammed Pervez, Macclesfield (GB); Ronald John Roberts, Macclesfield (GB); Christopher John Squire, Macclesfield (GB); Richard Anthony Storey, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 12/158,275

(22) PCT Filed: Dec. 18, 2006
(86) PCT No.: PCT/GB2006/004731

§ 371 Date: Jun. 19, 2008

Related U.S. Application Data
(60) Provisional application No. 60/752,831, filed on Dec. 21, 2005.

(51) Int. Cl. A61K 31/4184; C07D 235/06; A61P 35/00

(52) U.S. Cl. 514/394; 548/309.7

(57) ABSTRACT

The present invention relates to a tosylate salt of Compound 1 and polymorphs thereof, in particular crystalline and amorphous forms of Compound 1 tosylate salt, and methods of preparation thereof. Pharmaceutical compositions containing these salts as active ingredient, their use in the manufacture of medicaments for use in the treatment and/or prophylaxis of proliferative disease states, such as cancer, in the human or animal body, and their use in methods for the treatment and/or prophylaxis of proliferative disease states, such as cancer, in the human or animal body are also described.

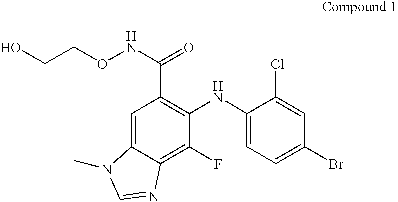

The present invention relates to a novel salt and, more particularly, to a novel salt of 6-(4-bromo-2-chloro-phenylamino)-7-fluoro-3-methyl-3H-benzoimidazole-5-carboxylic acid (2-hydroxy-ethoxy)-amide (hereinafter referred to as “Compound 1”), which is a MEK inhibitor that is useful in the treatment and/or prophylaxis of proliferative disease states, such as cancer, in the human or animal body. More specifically, the present invention relates to a tosylate salt of Compound 1 and to processes for the preparation of said salt. Also provided are pharmaceutical compositions containing a tosylate salt of Compound 1, as well as the use of the salt in the manufacture of medicaments for treatment and/or prophylaxis of proliferative disease states, such as cancer, in the human or animal body and methods of treating proliferative disease states, such as cancer, in a mammal by administering a therapeutically effective amount of a tosylate of Compound 1.

Cell signaling through growth factor receptors and protein kinases is an important regulator of cell growth, proliferation and differentiation. In normal cell growth, growth factors, through receptor activation (i.e., PDGF or EGF and others), activate MAP kinase pathways. One of the most important and most well understood MAP kinase pathways involved in normal and uncontrolled cell growth is the Ras/Raf kinase pathway. Active GTP-bound Ras results in the activation and indirect phosphorylation of Raf kinase. Raf then phosphorylates MEK1 and 2 on two serine residues (S218 and S222 for MEK1 and S222 and S226 for MEK2) (Ahn et al., Methods in Enzymology, 2001, 332:417-431). Activated MEK then phosphorylates its only known substrates, the MAP kinases ERK1 and 2. ERK phosphorylation by MEK occurs on Y204 and T202 for ERK1 and Y185 and T183 for ERK2 (Ahn et al., Methods in Enzymology 2001, 332:417-431). Phosphorylated ERK dimerizes and then translocates to the nucleus where it accumulates (Khokhlatchev et al., Cell 1998, 93:605-615). In the nucleus, ERK is involved in several important cellular functions, including but not limited to nuclear transport, signal transduction, DNA repair, nucleosome assembly and translocation, and mRNA processing and translation (Ahn et al., Molecular Cell, 2000, 6:1343-1354). Overall, treatment of cells with growth factors leads to the activation of ERK1 and 2 which results in proliferation and, in some cases, differentiation (Lewis et al., Adv. Cancer Res. 1998, 74: 49-139).

In proliferative diseases, genetic mutations and/or overexpression of the growth factor receptors, downstream signaling proteins, or protein kinases involved in the ERK kinase pathway lead to uncontrolled cell proliferation and, eventually, tumor formation. For example, some cancers contain mutations which result in the continuous activation of this pathway due to continuous production of growth factors. Other mutations can lead to defects in the deactivation of the activated GTP-bound Ras complex, again resulting in activation of the MAP kinase pathway. Mutated, oncogenic forms of Ras are found in 50% of colon and >90% pancreatic cancers as well as many others types of cancers (Kohl et al., Science, 1993, 260:1834-1837). Recently, bRaf mutations have been identified in more than 60% of malignant melanoma (Davies, H., et al., Nature 2002, 417:949-954). These mutations in bRaf result in a constitutively active MAP kinase cascade. Studies of primary tumor samples and cell lines have also shown constitutive or overactivation of the MAP kinase pathway in cancers of pancreas, colon, lung, ovary and kidney (Hoshino, R., et al., Oncogene 1999, 18:813-822). Hence, there is a strong correlation between cancers and an overactive MAP kinase pathway resulting from genetic mutations.

As constitutive or overactivation of MAP kinase cascade plays a pivotal role in cell proliferation and differentiation, inhibition of this pathway is believed to be beneficial in hyperproliferative diseases. MEK is a key player in this pathway as it is downstream of Ras and Raf. Additionally, it is an attractive therapeutic target because the only known substrates for MEK phosphorylation are the MAP kinases, ERK1 and 2. Inhibition of MEK has been shown to have potential therapeutic benefit in several studies. For example, small molecule MEK inhibitors have been shown to inhibit human tumor growth in nude mouse xenografts, (Sebolt-Leopold et al., Nature-Medicine 1999, 5(7):810-816; Trachet et al., AACR Apr. 6-10, 2002, Poster #5426; Tecle, H., IBC 2^{nd} International Conference of Protein Kinases, Sep. 9-10, 2002), block static allodynia in animals (WO 01/05390) and inhibit growth of acute myeloid leukemia cells (Milella et al., J. Clin. Invest. 2001, 108 (6):851-859).

Small molecule inhibitors of MEK have been disclosed. At least thirteen patent applications have appeared in the last several years: U.S. Pat. No. 5,525,625; WO 98/43960; WO 99/01421; WO 99/01426; WO 00/41505; WO 00/42002; WO 00/42003; WO 00/41994; WO 00/42022; WO 00/42029; WO 00/68201; WO 01/68619; and WO 02/06213.

Inhibitors of the MEK are also described in WO 03/077914. 6-(4-Bromo-2-chloro-phenylamino)-7-fluoro-3-methyl-3H-benzoimidazole-5-carboxylic acid (2-hydroxy-ethoxy)-amide, or “Compound 1”, is exemplified in WO 03/077914 and possesses the following structural formula:

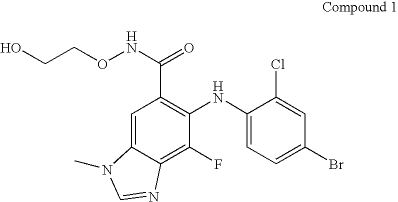

Compound 1 has been shown to possess inhibitory activity against MEK and therefore to be useful in the treatment of a hyperproliferative disease such as cancer.

WO 03/077914 discloses, in general terms, certain pharmaceutically acceptable salts of the compounds disclosed therein. Specifically, it is stated in WO 03/077914 that pharmaceutically acceptable salts of the compounds disclosed therein that possess a sufficiently basic moiety may form acid addition salts containing pharmaceutically acceptable anions, and a range of such anions are listed. Similarly, suitable salts of the compounds possessing an acidic moiety are to be formed by treatment of a compound with a basic compound and particularly an inorganic base.

The form of a pharmaceutically active compound which is used in medicaments is suitably one that provides for reasonable handling properties, which allow it to be processed and formulated. However, it is also necessary to ensure that the biological properties of the final formulation, such as dissolution rate of tablets and bioavailability of active ingredient are optimized, and there is frequently compromises to be made in selecting a particular form which best fulfils all these various requirements. However, in some cases, salts do not form easily and/or are not stable, which is probably due to low pKa values. The pKa value expresses the strength of acids and base, i.e., the tendency for an acid to lose a proton or a base to add a proton (Bronsted J. N., Rec. Trav. Chim. (1923) 47:718). This is particularly true for Compound 1.

The present invention provides a para toluene sulfonic acid (tosylate) salt of Compound 1 and various forms thereof, all of which are included within the scope of the invention, These forms include anhydrous forms as well as polymorphs and different stoichiometries of the salt. Further, the present invention provides a tosylate salt form of Compound 1 which shows unique physical and pharmaceutical properties that make it particularly suitable for use in medicaments.

In a further aspect the present invention provides a method of using a tosylate salt of Compound 1 as a medicament to treat a hyperproliferative disease or condition.

An additional aspect of the invention is the use of a tosylate salt of Compound 1 in the preparation of a medicament for the treatment or prevention of a hyperproliferative disease or condition.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

The accompanying drawings, which are incorporated herein and form a part of the specification, illustrate non-limiting embodiments of this invention, and together with the description, serve to explain the principles of the invention.

### IN THE FIGURES

FIG. 1 SHOWS THE XRPD OF THE 1:1 stoichiometry (Compound 1:counterion) tosylate salt of Compound 1 polymorph Form 1;

FIG. 2 shows the XRPD of the 1:1 stoichiometry (Compound 1:counterion) tosylate salt of Compound 1 polymorph Form 2;

FIG. 3 shows the XRPD of the 2:1 stoichiometry (Compound 1:counterion) tosylate salt of Compound 1;

FIG. 4 shows the DSC of the 1:1 stoichiometry (Compound 1:counterion) tosylate salt of Compound 1 polymorph Form 1;

FIG. 5 shows the DSC of the 1:1 stoichiometry (Compound 1:counterion) tosylate salt of Compound 1 polymorph Form 2;

FIG. 6 shows the XRPD of the 1:1 stoichiometry (Compound 1:counterion) tosylate salt of Compound 1 polymorph Form 2 after micronisation;

FIGS. 7-9 show the XRPD of the 1:1 stoichiometry (Compound 1:counterion) tosylate salt of Compound 1 during a disproportionation study in a buffer pH 6.5, at 0, 15 minutes and 60 minutes;

FIG. 10 shows the XRPD of Compound 1 free base; and

FIG. 11 is a graph showing the mean plasma concentration profiles for Compound 1 seen after oral dosing of dogs as described hereinafter.

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

The present invention provides a para toluene sulfonic acid (tosylate) salt of Compound 1 and various forms thereof, all of which are included within the scope of the invention. These forms include anhydrous forms as well as polymorphs and different stoichiometries of the salt. Depending upon the solvent and conditions for crystallisation, the compound can form salts with p-toluenesulfonic acid in various different stoichiometries. In one embodiment, the salt is in anhydrous form in the stoichiometry 1:1 (drug:counterion). In a particular embodiment, the salt is in the anhydrous form in the stoichiometry 1:1 (drug:counterion) and present as polymorph Form 1 or polymorph Form 2 as defined hereinafter. When crystallised from solvents as described herein, a 2:1 (drug to counterion) salt was formed, which appears to be an unusual feature of this salt. The formation of a 2:1 salt may be beneficial because it has a reduced counter ion load. Other solvents which are not described herein may also produce a 2:1 salt. Further, the present invention provides a tosylate salt form of Compound 1 which shows unique physical and pharmaceutical properties that make it particularly suitable for use in medicaments.

In certain embodiments, salts of Compound 1 are crystalline. The crystalline salts have been found to be better than the free base in terms in their handling properties from a manufacturing point of view, in particular their static and flow properties. The formation of salts may provide a means of purification, as process impurities can be separated and salts are generally easier to isolate than the free base.

In certain embodiments the tosylate salt of Compound 1 is a crystalline salt, which has surprisingly been found to possess improved pharmaceutical properties when compared to Compound 1 free base. In particular the dissolution rate of this salt has been found to be high, as well as its bioavailability as compared to the free base, as illustrated in the examples hereinafter.

When it is stated that the present invention relates to salts of Compound 1 that are crystalline salts the degree of crystallinity is conveniently greater than about 60%, more conveniently greater than about 80%, preferably greater than about 90% and more preferably greater than about 95%. Most preferably the degree of crystallinity is greater than about 98%.

The extent of enhanced bioavailability offered by the tosylate salt is surprising and is particularly useful, since the free base of Compound 1 has been classified as a BCS Class 4 compound. BCS Class 4 compounds normally have low bioavailability due to both low dissolution rate and permeability, and the limitation of permeability on absorption means that such salts would not usually be expected exert a substantial impact on absorption (See for example: Dressman et al. (2001) Pharm Tech. July: 68).

The behaviour of the tosylate salt of Compound 1 in formulations is interesting. Form 2 of the tosylate salt of Compound 1 appears to have some kinetic resistance to disproportionate in suspension in buffered media, whereas other salts investigated readily disproportionate to the free form in aqueous environments in a similar timescale. The tosylate salt also shows a unique resistance to becoming amorphous on milling and micronisation, a property not observed with other salts. These properties appear to be unique to this particular salt and may explain the enhanced pharmaceutical effects noted and illustrated hereinafter.

In particular, the one embodiment of the salt is anhydrous tosylate salt of Compound 1 with a stoichiometry 1:1. Two polymorphs of this anhydrous salt have been identified (see Examples hereinafter) and are designated as “Form 1” and “Form 2”. In one embodiment, the salt is in the form of the polymorph Form 2. In another embodiment, the salt is in the form of the polymorph Form 1. It has been found that the salt in the form of the polymorph Form 2 is more stable than the salt in the form of the polymorph Form 1.

Preparation of the salt can be effected by reacting a slurry of Compound 1 in an organic solvent with at least a stoichiometric amount of para-toluene sulfonic acid. Thus in a further aspect, the invention provides a method for preparing the tosylate salt of Compound 1, said method comprising:

(i) reacting a slurry of Compound 1 in an organic liquid with toluene sulfonic acid; and

(ii) precipitating the salt from the resultant solution.

The mole ratio of the amount of Compound 1:toluene sulfonic acid for the manufacture of the 1:1 stoichiometry salt is suitably in the range of from 0.95:1 to 1.05:1, and is suitably a stoichiometric amount of 1:1. In certain embodiments, the salts obtained have a 1:1 stoichiometry of Compound 1:counterion, although under certain circumstances, as illustrated hereinafter, salts having stoichiometry of 2:1 of drug (Compound 1):counterion can be obtained. The reason for the existence of certain forms, in particular the 2:1 salt, is not fully understood, as Compound 1 appears to have only one ionizable center and the formation of hemi-salts with monoprotic acids is very unusual.

Step (i) is suitably carried out at a wide range of temperatures for, example from 20-100° C., as a further example at moderate temperatures such as from 20-40° C., and as a further example ambient temperature may be utilized. In an alternative embodiment elevated temperatures, such as from 60-100° C. and conveniently the reflux temperature of the solvent may be used. During step (i), Compound 1 and the counterion readily dissolve into solution. The salt formed during this reaction is readily precipitated.

Suitable organic liquids include organic solvents in which Compound 1 and its salts are sparingly soluble. As used herein, the expression “sparingly soluble” means having a solubility less than 100 mL of solvent per gram of solute, for example between 30 and 100 mL of solvent per gram of solute. These solvents include (i) alcohols, for example C₁₋₆ alcohols such as methanol, ethanol or isopropanol, (ii) alkyl ketones, for example C₁₋₆ alkyl ketones such as 2-butanone, and (iii) esters such as C₁₋₆ alkyl esters, for example ethyl acetate. In a particular embodiment, the organic solvent is a C₁₋₆ alcohol such as methanol or isopropanol.

In one embodiment, the amount of solvent used in step (i) is an amount sufficient to allow some dissolution, for instance substantially complete or complete dissolution of Compound 1 free base and the counter-ion to occur. Generally, the amount of Compound 1:organic liquid is in the range of from 1:5-1:100 w/v.

The particular polymorphic form of the salt obtained or the stoichiometry of the salt may vary depending upon the precise conditions used in steps (i) and (ii). For instance, it has been found that polymorphic Form 2 as defined herein may be obtained at moderate temperatures as described above and with relatively low volumes of solvent, for example of from 1:5 to 1:10 w/v of Compound 1:organic liquid. A particular organic liquid in this case may be methanol. Polymorphic Form 1 has been obtained when step (i) is conducted at elevated temperatures as defined above and with higher volumes of solvent (for example from 1:40-1:60 w/v of Compound 1:organic liquid, which may be, for example, isopropanol). Examples of conditions under which Compound 1 tosylate salt with a 2:1 stoichiometry are illustrated in Example 3.

Seeding of the solution with the tosylate salt of Compound 1 crystals, suitably in the required polymorphic form or stoichiometry, at this stage may assist in the precipitation process of step (ii).

Suitably, after step (ii) the precipitate is separated from the organic liquid by filtration. The recovered precipitate is optionally washed, for example with the same organic liquid as used in step (i) and dried, for example at elevated temperature, for example of from 30-60° C., such as from 40-50° C., under reduced pressure to a constant weight to yield the desired salt.

The physical properties of the tosylate salts obtained by the methods of this invention were investigated and are described in the Examples.

The invention also includes isotopically-labeled compounds, which are identical to those recited in the present invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F and ³⁶Cl, respectively. The tosylate salt of Compound 1 and polymorphs thereof which contain the aforementioned isotopes and/or other isotopes of other atoms are within the scope of this invention. Certain isotopically-labeled compounds of the present invention, for example those into which radioactive isotopes such as ¹H and ¹⁴C are incorporated, are useful in drug and/or substrate tissue distribution assays. Tritiated, i.e., 3H and carbon-14, i.e., ¹⁴C, isotopes are particularly widely used as a result of their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be utilized in some particular circumstances. Isotopically labeled salts of the present invention can generally be prepared by carrying out procedures disclosed in WO 03/077914 by substituting a readily available isotopically labeled reagent for a non-isotopically labeled reagent during the preparation, or if desired, using an isotopically labeled sulfuric acid in the preparation of the salt,

A further aspect of the invention provides a pharmaceutical composition which comprises a Compound 1 tosylate salt as defined herein in association with a pharmaceutically acceptable excipient or carrier. The composition may be in a form suitable for oral administration (for example as tablets, lozenges, hard or soft capsules, emulsions, dispersible powders or granules, syrups, elixirs or oily or extemporaneously prepared aqueous suspensions), for administration by inhalation (for example as a finely divided powder or a liquid aerosol), for administration by insufflation (for example as a finely divided powder), for parenteral injection (for example as a sterile solution, suspension or emulsion for intravenous, subcutaneous, intramuscular, intravascular or infusion dosing), for topical administration (for example as creams, ointments, gels, oily solutions or suspensions or extemporaneously prepared aqueous suspensions), or for rectal administration (for example as a suppository). In a particular embodiment, Compound 1 tosylate salt is administered orally. In general the above compositions may be prepared in a conventional manner using conventional excipients.

The compositions of the present invention are advantageously presented in unit dosage form. Tablet dosage forms are particular embodiments. Thus, according to a further aspect of the present invention there is provided a tablet comprising Compound 1 tosylate salt as defined herein in association with a pharmaceutically acceptable excipient or carrier.

The amount of the active compound administered will be dependent on the subject being treated, the severity of the disorder or condition, the rate of administration, the disposition of the compound and the discretion of the prescribing physician. However, an effective dosage is in the range of about 0.01 to about 100 mg per kg body weight per day, preferably about 1 to about 35 mg/kg/day, in single or divided doses. For a 70 kg human, this would amount to about 0.7 to 7000 mg/day, preferably about 70 to about 2500 mg/day. In some instances, dosage levels below the lower limit of the aforesaid range may be more than adequate, while in other cases still larger doses may be employed without causing any harmful side effect, provided that such larger doses are first divided into several small doses for administration throughout the day. A unit dosage form such as a tablet or capsule will usually contain, for example 1-1000 mg of active ingredient, and preferably 5-420 mg of active ingredient. Preferably a daily dose in the range of 0.03-6 mg/kg is employed.

A further aspect of the present invention provides a Compound 1 tosylate salt as defined herein for use in a method of treatment or prophylaxis of the human or animal body by therapy. Yet another aspect of the present invention provides a tosylate salt of Compound 1 as defined herein for use as a medicament. In a further aspect, the present invention provides a tosylate salt of Compound 1 as defined herein for use as a medicament for the treatment of disease states mediated through NMK, in particular proliferative disorders, or abnormal cell growth, such as cancer, in a warm-blooded mammal such as a human.

According to a further aspect of the invention there is provided the use of a tosylate salt of Compound 1 as defined herein in the manufacture of a medicament for use in the treatment of disease states mediated through the MEK, in particular proliferative disorders, or abnormal cell growth, such as cancer, in a warm-blooded mammal such as a human.

According to a further feature of the invention there is provided a method for treating disease states mediated through the MEK, in particular proliferative disorders, or abnormal cell growth, such as cancer, in a warm-blooded mammal, such as a human, in need of such treatment which comprises administering to said mammal an effective amount of a tosylate salt of Compound 1 as herein before defined, or a pharmaceutical composition thereof.

Particular examples of proliferative disorders, which may be treated using the salts or compositions of the invention, include hyperproliferative disorders in a mammal. Particular cancers are brain, lung, squamous cell, bladder, gastric, pancreatic, breast, head, neck, renal, kidney, ovarian, prostate, colorectal, esophageal, testicular, gynecological or thyroid cancer.

However, the compounds and compositions of the invention may also be used in the treatment of a non-cancerous hyperproliferative disorder such as benign hyperplasia of the skin (e.g., psoriasis), restenosis, or prostate (e.g., benign prostatic hypertrophy (BPH)).

Other examples of MEK mediated diseases, which may be treated using the compounds, or compositions of the invention include pancreatitis or kidney disease (including proliferative glomerulonephritis and diabetes-induced renal disease) or the treatment of pain in a mammal.

The compounds and compositions may also be used for the prevention of blastocyte implantation in a mammal, or for treating a disease related to vasculogenesis or angiogenesis in a mammal. Such diseases may include tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The terms “abnormal cell growth” and “hyperproliferative disorder” are used interchangeably in this application and refer to cell growth that is independent of normal regulatory mechanisms (e.g., loss of contact inhibition). This includes, for example, the abnormal growth of: (1) tumor cells (tumors) that proliferate by expressing a mutated tyrosine kinase or over expression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment” as used herein, unless otherwise indicated, refers to the act of treating as “treating” is defined immediately above.

Thus patients that can be treated with compounds or compositions of the present invention include, for example, patients that have been diagnosed as having psoriasis, restenosis, atherosclerosis, BPH, lung cancer, non small cell lung cancer, bone cancer, CMML, pancreatic cancer, colorectal, skin cancer, cancer of the head and neck, melanoma (in particular cutaneous or intraocular melanoma), uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), ovarian cancer, multiple myeloma, hepatocellular carcinoma, Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, in particular acute myeloid leukaemia solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The tosylate salt of Compound 1 may be applied as a sole therapy or may involve, in addition to the tosylate salt of Compound 1, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to Compound 1 tosylate salt may be surgery, radiotherapy or chemotherapy. Such chemotherapy may cover categories of therapeutic agent such as:

(i) antiangiogenic agents such as those which inhibit the effects of vascular endothelial growth factor, (for example the anti-vascular endothelial cell growth factor antibody bevacizumab [Avastin™], and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and those that work by different mechanisms from those defined herein (for example linomide, inhibitors of integrin αvβ3 function, angiostatin, razoxin, thalidomide, MMP-2 (matrix-metalloprotienase 2) inhibitors, MMP-9 (matrix-metalloprotienase 9) inhibitors, and COX-II (cyclooxygenase II) inhibitors), and

(ii) vascular targeting agents (for example combretastatin phosphate and compounds disclosed in WO 00/40529, WO 00/41669, WO 01/92224, WO 02/04434 and WO 02/08213, and the vascular damaging agents described in International Patent Application Publication No. WO 99/02166, (for example N-acetylcolchinol-O-phosphate));

(iii) cytostatic agents such as antioestrogens (for example tamoxifen, toremifene, raloxifene, droloxifene, and iodoxyfene), oestrogen receptor down regulators (for example fulvestrant), progestogens (for example megestrol acetate), aromatase inhibitors (for example anastrozole, letrazole, vorazole, and exemestane), antiprogestogens, antiandrogens (for example flutamide, nilutamide, bicalutamide, and cyproterone acetate), LHRH agonists and antagonists (for example goserelin acetate, leuprorelin, and buserelin), inhibitors of 5α-reductase (for example finasteride);

(iv) anti-invasion agents (for example metalloproteinase inhibitors like marimastat and inhibitors of urokinase plasminogen activator receptor function or antibodies to Heparanese;

(v) inhibitors of growth factor function (such growth factors include for example platelet derived growth factor and hepatocyte growth factor), such inhibitors include growth factor antibodies, growth factor receptor antibodies, (for example the anti-erbb2 antibody trastuzumab [Herceptin™], the anti-EGFR antibody panitumumab, the anti-erbB1 antibody cetuximab [C225]), and any growth factor or growth factor receptor antibodies disclosed by Stern et al. Critical reviews in oncology/haematology, 2005, Vol. 54, pp 11-29); such inhibitors also include tyrosine kinase inhibitors such as inhibitors of the epidermal growth factor family (for example EGFR family tyrosine kinase inhibitors such as N-(3-chloro-4-fluorophenyl)-7-methoxy-6-(3-morpholinopropoxy)-quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy) quinazolin-4-amine (CI 1033)) and erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

vi) antiproliferative/antineoplastic drugs and combinations thereof, as used in medical oncology, such as antimetabolites (for example antifolates such as methotrexate, fluoropyrimidines such as 5-fluorouracil, tegafur, purine and adenosine analogues, and cytosine arabinoside, hydroxyurea or, for example, one of the anti-metabolites specifically disclosed in European Patent Application No. 239362 such as N-(5-[N-(3,4-dihydro-2-methyl-4-oxoquinazolin-6-ylmethyl)-N-methylamino-2-thenoyl)-L-glutamic acid; antitumour antibiotics (for example anthracyclines such as adriamycin, bleomycin, doxorubicin, daunomycin, epirubicin and idarubicin, mitomycin-C, dactinomycin, and mithramycin); platinum derivatives (for example cisplatin, and carboplatin); alkylating agents (for example nitrogen mustard, melphalan, chlorambucil, busulphan, cyclophosphamide, ifosfamide, nitrosoureas, and thiotepa); antimitotic agents (for example vinca alkaloids such as vincristine, vinblastine, vindesine, and vinorelbine, and taxoids such as taxol and taxotere); topoisomerase inhibitors (for example epipodophyllotoxins such as etoposide and teniposide, amsacrine, topotecan, camptothecin and irinotecan); enzymes (for example asparaginase); and thymidylate synthase inhibitors (for example raltitrexed);

and additional types of chemotherapeutic agent including:

(vii) biological response modifiers (for example interferon);

(viii) antibodies (for example edrecolomab);

(ix antisense therapies, for example those which are directed to the targets listed above, such as ISIS 2503, an anti-ras antisense;

(x) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(xi) immunotherapy approaches, including for example ex-vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

For example, a tosylate salt of Compound 1 may be used in conjunction with an effective amount of one or more substances selected from anti-angiogenesis agents, signal transduction inhibitors, and antiproliferative agents. In a particular embodiment, anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a Compound 1 tosylate of the present invention and pharmaceutical compositions described herein. Examples of useful COX-II inhibitors include CELBREX™ (alecoxib), valdecoxib, and rofecoxib. Examples of useful matrix metalloprotienase inhibitors are described in WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, U.S. Pat. No. 5,863,949, and U.S. Pat. No. 5,861,510, all of which are incorporated herein in their entireties by reference. Suitable MMP-2 and MMP-9 inhibitors are those that have little or no activity inhibiting MMP-1. In particular, those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13) are used. Particular examples of MMP inhibitors useful in the present invention are AG-3340, RO 32-3555, and RS 13-0830.

Therefore, a further aspect of the present invention the tosylate salt of Compound 1 in combination with any one of the anti tumour agents listed under (i)-(xi) herein above. A further aspect of the present invention provides the tosylate salt of Compound 1 in combination with one or more of the anti tumour agents listed under (i)-(xi) herein above. A further aspect of the present invention provides the tosylate salt of Compound 1 in combination with any one of the classes of anti-tumour agents listed under (i)-(xi) herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the present invention there is provided a kit comprising the tosylate salt of Compound 1 in combination with an anti-tumour agent selected from one listed under (i)-(xi) herein above.

According to a further aspect of the present invention there is provided a kit comprising:

a) the tosylate salt of Compound 1 in a first unit dosage form;

b) an anti-tumour agent selected from one listed under (i)-(xi) herein above; in a second unit dosage form; and

c) container means for containing said first and second dosage forms.

Compound 1 has been found to have activity in the following assay. N-terminal 6 His-tagged, constitutively active MEK1 (2-393) is expressed in E. coli and protein is purified by conventional methods (Ahn et al., Science 1994, 265, 966-970). The activity of MEK1 is assessed by measuring the incorporation of γ-³³P-phosphate from γ-³³P-ATP onto N-terminal His tagged ERK2, which is expressed in E. coli and is purified by conventional methods, in the presence of MEK1. The assay is carried out in 96-well polypropylene plate. The incubation mixture (100 μL) comprises of 25 mM Hepes, pH 7.4, 10 mM MgCl₂, 5 mM β-glycerolphosphate, 100 μM sodium orthovanadate, 5 mM DTT, 5 nM MEK1, and 1 μM ERK2. Inhibitors are suspended in DMSO, and all reactions, including controls are performed at a final concentration of 1% DMSO. Reactions are initiated by the addition of 10 μM ATP (with 0.5 μCi γ-³³P-ATP/well) and incubated at ambient temperature for 45 minutes. Equal volume of 25% TCA is added to stop the reaction and precipitate the proteins. Precipitated proteins are trapped onto glass fiber B filterplates, and excess labeled ATP washed off using a Tomtec MACH III harvestor. Plates are allowed to air-dry prior to adding 30 μL/well of Packard Microscint 20, and plates are counted using a Packard TopCount. In this assay, compounds of the invention exhibited an IC₅₀ of less than 50 micromolar.

### EXAMPLES

In order to illustrate the invention, the following examples are included. However, it is to be understood that these examples do not limit the invention and are only meant to suggest a method of practicing the invention. Yields are given for the Examples as performed and could likely be enhanced through further development.

### Example 1

### Preparation of Tosylate Salt of Compound 1

### Form 2

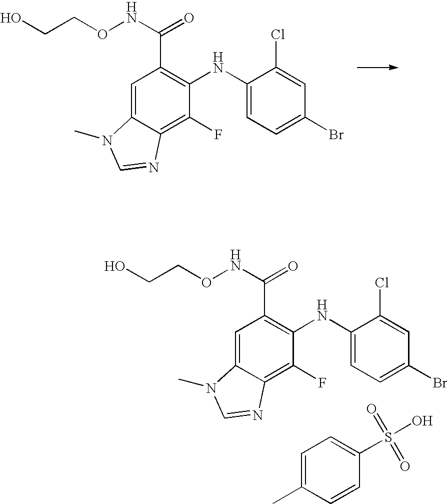

To a stirred suspension of 6-(4-bromo-2-chloro-phenylamino)-7-fluoro-3-methyl-3H-benzoimidazole-5-carboxylic acid (2-hydroxy-ethoxy)-amide (15.20 g, 33.21 mmol) (obtainable as described in Example 10 of WO 03/077914, which is incorporated herein by reference) in methanol (92 mL) at ambient temperature was added a solution of para-toluenesulfonic acid monohydrate (6.71 g, 34.92 mmol) in methanol (61 mL). The slurry dissolved to give a brown solution, which rapidly precipitated. The resulting slurry was stirred at ambient temperature for 2.5 hours. The slurry was filtered, washed with methanol (15.2 mL) and dried under reduced pressure at 40° C. to a constant weight to give 6-(4-bromo-2-chloro-phenylamino)-7-fluoro-3-methyl-3H-benzoimidazole-5-carboxylic acid (2-hydroxy-ethoxy)-amide p-toluenesulfonate (19.71 g, 29.60 mol, 89% yield) as a yellow crystalline solid as the 1:1 stoichiometry polymorph Form 2.

¹H NMR (400 MHz, D₆ DMSO; with added tetramethylsilane (TMS) for reference) δ 2.29 (3H, s, CH₃), 3.57 (2H, t, CH₂OH), 3.89 (2H, t, CH₂ON), 3.98 (3H, s, CH₃), 6.09 (2H, br, OH, NH), 6.47 (1H, dd, ArH), 7.11 (2H, d, ArH), 7.28 (1H, dd, ArH), 7.48 (2H, d, ArH), 7.63 (1H, d, ArH), 7.91 (1H, s, ArH), 8.10 (1H, br, ArNH), 8.96 (1H, s, NNCH), 11.86 (1H, br, ONH). ¹³C NMR (100 MHz, D₆ DMSO) δ 20.8 (CH₃), 32.1 (CH₃), 58.5 (CH₂OH), 77.3 (CH₂ON), 108.3 (CH), 108.3 (CC═O), 109.7 (CBr), 115.8 (CH), 115.8 (CH), 120.6 (CCl), 122.1 (C), 125.1 (C), 125.4 (2×CH), 128.1 (2×CH), 130.5 (C), 131.1 (CH), 132.2 (C), 137.8 (CF), 140.6 (C), 144.5 (C═O), 145.3 (C), 146.4 (CH).

### Example 2

### Preparation of Form 1 Polymorph of the Salt Obtained in Example 1

To a slurry of Compound 1 (0.5 g) in isopropanol (5 mL, 10 volumes) was added p-toluene sulfonic acid monohydrate (0.21 g, 1.00 mol) and the mixture was heated to reflux. An almost complete solution was obtained. A further 2.5 mL of isopropanol was added but further solubilization did not occur. Within a couple of minutes it was noticed that another solid had precipitated out of solution. On further heating the solid did not redissolve. The slurry was cooled to 20° C., and the solid was filtered and then dried in the vacuum oven at 50° C. overnight.

### Example 3

### Preparation of a 2:1 Stoichiometry Tosylate Salt

### Compound 1: Counterion

### Preparation A

To a slurry of Compound 1 (0.5 g) in NMP (2 mL) and isopropanol (5 mL) was added p-toluene sulfonic acid monohydrate (0.21 g, 1.00 mol) and the mixture was heated to 75° C. At this point a complete solution was obtained. The solution was cooled to 20° C. and filtered, and then the solid was dried in a vacuum oven at 50° C. overnight.

### Preparation B

To a slurry of Compound 1 (0.5 g) in methanol (15 mL, 30 volumes) was added p-toluene sulfonic acid monohydrate (0.21 g, 1.00 mol). A complete solution was obtained at 20° C. The solution was stirred for 15 minutes and a precipitation noticed. The resultant slurry was heated to 60° C. but did not dissolve. The slurry was cooled to 20° C. and then filtered, and then the solid was dried in a vacuum oven at 50° C.

### Example 4

### Investigation into the Physical Properties of the Tosylate Salt

The products of Examples 1-3 were subject to the following tests to determine their physical properties.

### Powder X-Ray Diffraction (PXRD)

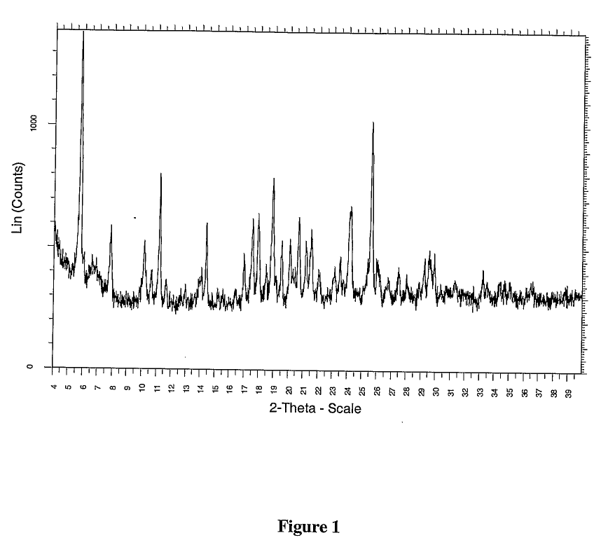

All samples were run on a Bruker D5000 diffractometer. The X-ray powder diffraction spectra were determined by mounting a sample of the crystalline salt on Siemens single silicon crystal (SSC) wafer mounts and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The running time was 31 minutes and 41 seconds. The instrument was equipped with a scintillation counter as detector. Control and data capture was by means of a Dell Optiplex 686 NT 4.0 Workstation operating with Diffract+software. Data were collected over the range 2-theta 2-40°, in increments of 2-theta 0.020 with 4 s per increment. The results are shown in FIGS. 1 to 3. The PXRD peak assignments for form 1 (FIG. 1) of tosylate salt (1:1 stoichiometry) are summarized in Table 1.

TABLE 1

| Angle | Intensity |
|---|---|
| (2-Theta °) | (%) |
| 5.75 | 100 |
| 2.76 | 89 |
| 25.60 | 73.7 |
| 11.17 | 57.8 |
| 18.89 | 55.5 |
| 3.67 | 54 |
| 24.18 | 47.4 |
| 17.93 | 46.1 |
| 20.68 | 45.3 |
| 17.53 | 44.5 |
| 14.38 | 43.2 |
| 7.82 | 42.2 |
| 21.52 | 41.7 |
| 19.50 | 38.2 |
| 21.17 | 38.1 |
| 10.13 | 37.8 |
| 20.09 | 37 |
| 29.58 | 35.8 |
| 29.90 | 34.7 |
| 4.23 | 34.5 |
| 16.97 | 34.3 |
| 6.01 | 34.1 |
| 26.04 | 32.7 |
| 23.49 | 32.2 |
| 25.33 | 31.4 |
| 18.46 | 31.2 |
| 29.22 | 31 |
| 6.60 | 30.9 |
| 27.48 | 30.6 |
| 23.12 | 30.6 |
| 33.23 | 30 |
| 4.87 | 30 |
| 22.06 | 29.6 |
| 10.61 | 29 |
| 7.08 | 28.4 |
| 26.79 | 27.7 |
| 14.01 | 27.4 |
| 34.7 | 27.1 |
| 31.31 | 26.8 |
| 33.53 | 26.6 |
| 34.36 | 26.6 |
| 35.08 | 26.6 |
| 11.61 | 26.4 |
| 36.49 | 26.3 |
| 28.86 | 26 |
| 28.07 | 25.9 |
| 30.37 | 25.8 |
| 30.76 | 25.4 |
| 38.83 | 25.3 |
| 36.21 | 25 |
| 25.12 | 24.4 |
| 31.85 | 24.3 |
| 35.37 | 24 |
| 12.91 | 24 |
| 22.77 | 23.8 |
| 37.98 | 23.8 |
| 16.39 | 23.7 |
| 12.64 | 23.5 |
| 39.27 | 23.2 |
| 8.20 | 22.6 |
| 15.20 | 21.9 |
| 15.49 | 21.2 |
| 15.97 | 20.4 |
| 12.06 | 20.1 |

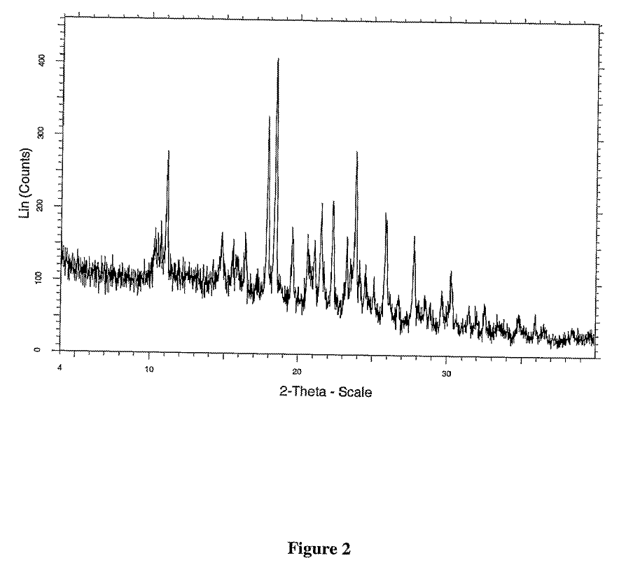

The PXRD peak assignments for Form 2 (FIG. 2) of tosylate salt (1:1 stoichiometry) are summarized in Table 2.

TABLE 2

| Angle | Intensity |
|---|---|
| (2-Theta °) | (%) |
| 18.43 | 100 |
| 23.85 | 64.9 |
| 17.86 | 64.6 |
| 25.91 | 50.5 |
| 11.08 | 47 |
| 27.78 | 43.7 |
| 22.30 | 41.6 |
| 21.49 | 32.1 |
| 23.25 | 31.9 |
| 16.38 | 30.2 |
| 20.61 | 29.4 |
| 19.57 | 28.8 |
| 10.29 | 28.7 |
| 30.31 | 28.4 |
| 15.81 | 28.1 |
| 2.86 | 26.5 |
| 21.07 | 25 |
| 2.67 | 24.5 |
| 28.53 | 24.5 |
| 14.77 | 24 |
| 29.66 | 23.9 |
| 21.7 | 23.6 |
| 26.73 | 22.8 |
| 25.11 | 22.5 |
| 2.5 | 22.4 |
| 32.54 | 22.1 |
| 10.50 | 21.9 |
| 2.16 | 21 |
| 15.52 | 20.9 |
| 23.48 | 20.6 |
| 24.54 | 20.4 |
| 34.90 | 20 |
| 31.43 | 20 |

The peak at 18.43° is particularly strong.

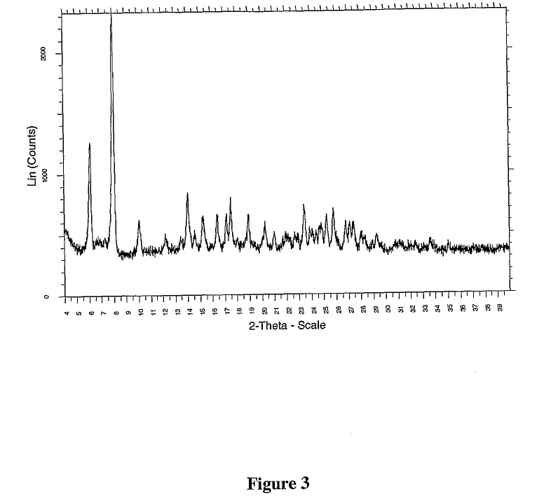

The PXRD peak assignments for the tosylate salt with a stoichiometry of Compound 1: tosylate of 2:1 (FIG. 3) is summarized in Table 3.

TABLE 3

| Angle | Intensity |
|---|---|
| (2-Theta °) | (%) |
| 7.96 | 100 |
| 2.60 | 53.9 |
| 2.36 | 53.9 |
| 6.07 | 53.8 |
| 2.89 | 52 |
| 14.00 | 36 |
| 17.48 | 34.2 |
| 3.6 | 32.8 |
| 23.45 | 31.1 |
| 25.80 | 30.1 |
| 16.37 | 28.6 |
| 18.89 | 28.3 |
| 25.24 | 28 |
| 15.23 | 27.9 |
| 17.11 | 27.7 |
| 3.84 | 26.9 |
| 10.04 | 26.7 |
| 20.25 | 25.7 |
| 26.78 | 25.6 |
| 27.42 | 25.4 |
| 27.11 | 25.4 |
| 24.72 | 24.3 |
| 4.17 | 23 |
| 14.55 | 23 |
| 24.08 | 22.9 |
| 23.90 | 22.8 |
| 24.39 | 22.5 |
| 21 | 22 |
| 28.04 | 21.7 |
| 22.92 | 21.5 |
| 22.14 | 21.3 |
| 29.30 | 20.9 |
| 22.68 | 20.9 |
| 7.26 | 20.6 |
| 21.72 | 20.3 |
| 21.96 | 20.1 |
| 18.01 | 20 |
| 12.21 | 19.8 |
| 28.33 | 19.5 |
| 13.46 | 19.5 |
| 33.64 | 18.8 |
| 28.97 | 18.6 |
| 32.45 | 18.3 |
| 35.09 | 18.2 |
| 30.75 | 18 |
| 29.64 | 17.5 |
| 31.32 | 16.9 |
| 31.97 | 16.7 |
| 37.04 | 15.8 |
| 37.80 | 15.7 |

Persons skilled in the art of X-ray powder diffraction will realise that the relative intenslty of peaks can be affected by, for example, grains above 30 microns in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values (see Jenkins, R. & Snyder, R. L., “Introduction to X-Ray Powder Diffractometry”, John Wiley & Sons, 1996, for further information).

### Differential Scanning Calorimetry

Differential Scanning Calorimetry (DSC) analysis was conducted on Compound 1 tosylate salts 1:1 using a Mettler DSC820e. Samples of typically less than 5 mg of material contained in a 40 μL aluminum pan fitted with a pierced lid were heated over the temperature range of 25° C. to 325° C. at a constant heating rate of 10° C. per minute. A purge gas using nitrogen was used with a flow rate of 100 mL per minute.

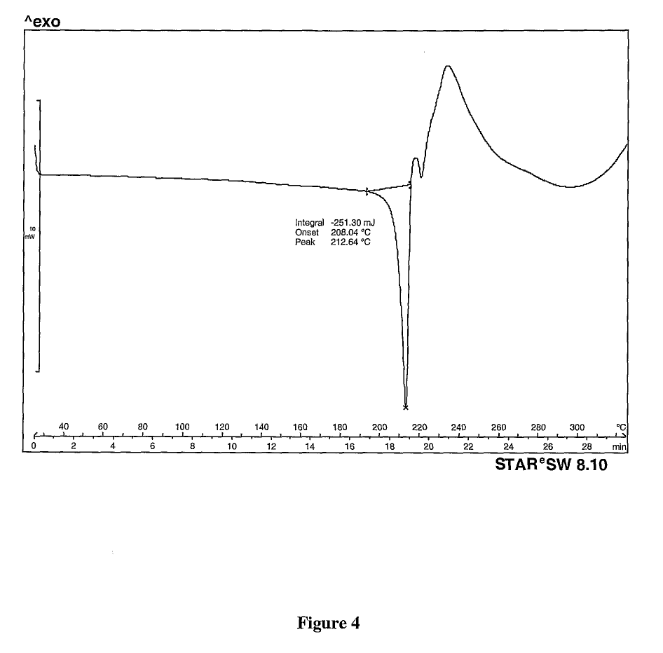

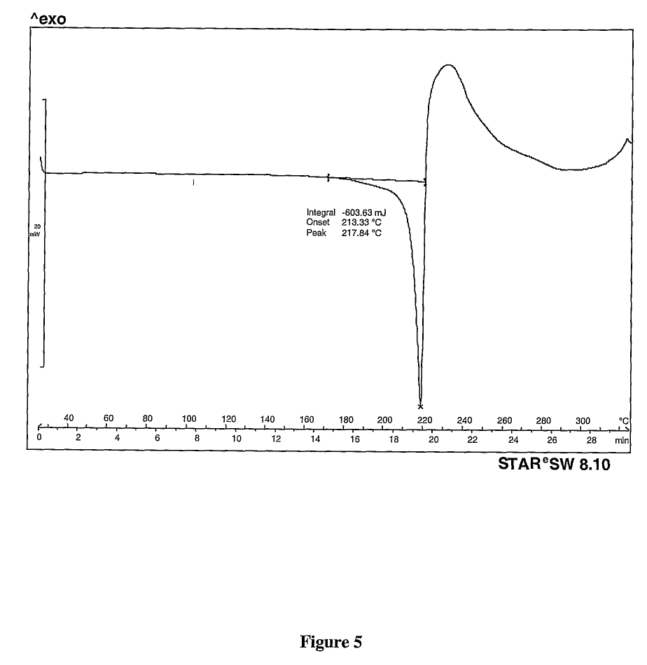

The results indicate that the polymorph Form 2 shows a large, sharp endotherm with a peak melting temperature of 218° C. due to melting FIG. 5), whereas polymorph Form 1 shows a large, sharp endotherm with a peak melting temperature of 213° C. There is a small amount of conversion of polymorph Form 1 to Form 2 on melting, hence a second event is seen following the melting of Form 1 in FIG. 4. It will be understood that the onset and/or peak temperature values of the DSC may vary slightly from one machine to another, one method to another or from one sample to another, and so the values quoted are not to be construed as absolute.

### Example 5

### Particle Size Reduction Investigations

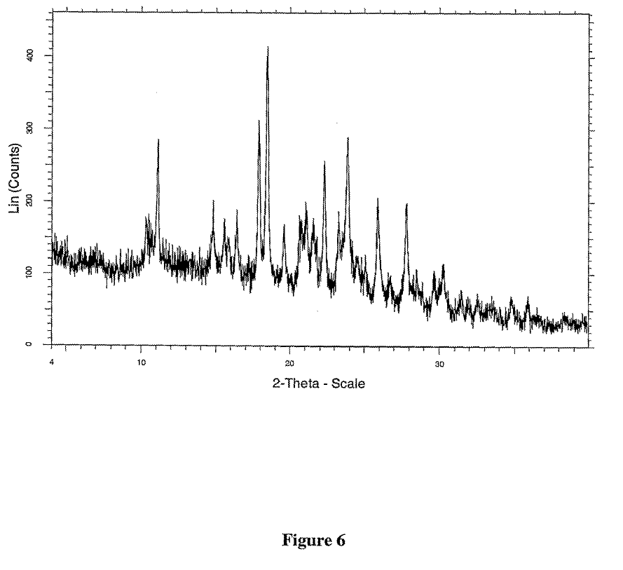

In order to investigate the potential for the materials to become amorphous during particle size reduction procedures, batches of the material were milled either by grinding the material in a pestle and mortar for 10 minutes or by micronising using conventional micronising equipment, in this case, a 2″ Spiral jet mill manufactured by Gravesend engineering. The products were subjected to PXRD as described in Example 4 above. Results shown in FIG. 6 indicate that the tosylate salt is highly resistant to becoming amorphous during these processes, a property not observed by other salts tested using these techniques.

### Example 6

### Solubility and Dissolution

In order to investigate the effects of salts on the release rate, an intrinsic dissolution (pH 6.5) was performed. The tosylate salt had a powder dissolution rate of 60 times higher than the dissolution rate of the free base, and an intrinsic dissolution rate comparable to that of the free base.

Due to the observation of the tosylate salt remaining as the salt form in solution, further investigations were performed to probe the solubility of the salts in various media. The amounts dissolved (mg/mL) in the various media after stirring either the Compound 1 free base or the Compound 1 tosylate salt with excess solid for 10 minutes and 60 minutes are shown in Table 4 (SIF=Simulated intestinal fluid; FaSIF=Fasted Simulated Intestinal Fluid).

TABLE 4

| | Compound 1 free base | | Compound 1 Tosylate | |
|---|---|---|---|---|
| Media | 10 min | 60 min | 10 min | 60 min |
| SIF pH 6.5 | 0.004 | 0.004 | 0.041 | 0.020 |
| Sorenson's buffer | 0.004 | 0.004 | 0.019 | 0.007 |
| pH 1.2 | 0.14 | 0.13 | 0.41 | 0.19 |
| FaSSIF | 0.01 | 0.01 | 0.19 | 0.04 |
| H₂O | 0.004 | N/D | 0.058 | N/D |

Dissolution of the free base of Compound 1 as well as the tosylate salt of Compound 1 was investigated at the 1 hour time point in both tablet form and powder form. The percent dissolutions after 1 hour are shown in Table 5. The dissolution rate of the tosylate salt was generally much higher than that of the free base.

TABLE 5

| | | Compound 1 | | Compound 1 |
|---|---|---|---|---|
| | Compound 1 | Free base | Compound 1 | tosylate |
| | Free base | (50 mg | tosylate | (50 mg fbe |
| | (powder) | tablets) | (powder) | tablets) |
| SIF pH 6.5 | 2.36 | 2.61 | 5.16 | 6.66 |
| FaSSIF | 4.49 | 4.54 | 31.48 | 23.05 |

### Example 7

### Disproportionation Studies

The characteristics of the salts in liquids were investigated by slurrying the material in a range of solvents including a buffer of pH 6.5 to simulate physiological conditions.

Compound 1 tosylate salt was slurried in pH 6.5 buffer at room temperature at a concentration of 100 mg in 2 mL. Samples were taken at 15 minute intervals up to 120 minutes. The results indicate that although a small amount of free base is produced after 15 minutes slurry the salt remains present up to 75 minutes in the slurry.

In order to prepare the pH 6.5 slurry of Compound 1 tosylate salt, a buffer was first prepared by producing a first solution (Solution A) by adding 90.8 mg of potassium dihydrogen phosphate to a conical 100 mL flask. Deionised water was added to mark ensuring all solid had dissolved. A second solution (Solution B) was prepared by adding 118.8 mg of disodium hydrogen phosphate to a conical 100 mL flask and deionised water was added to mark ensuring all solid had dissolved. Then 64 mL of solution A was mixed with 32 mL of solution B to form a pH 6.5 buffer

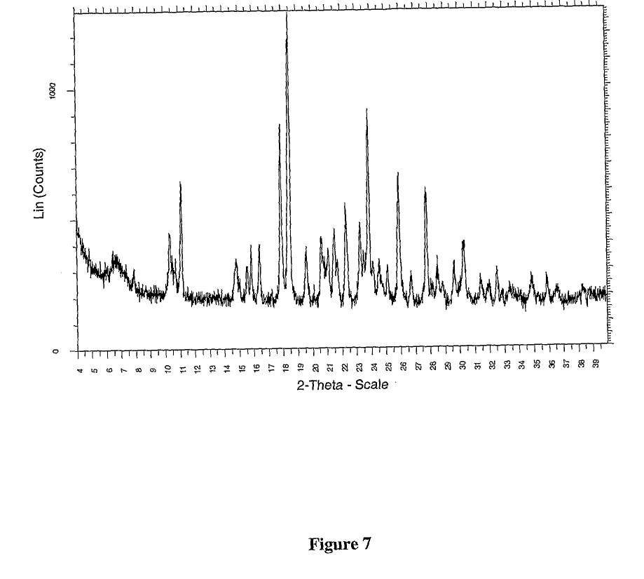

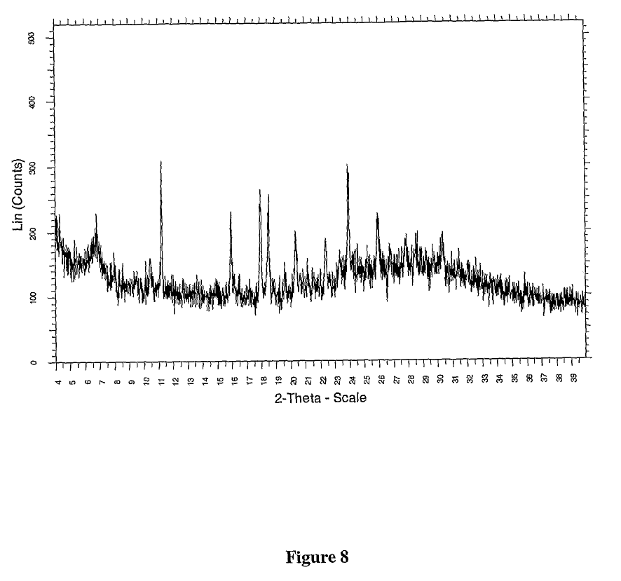

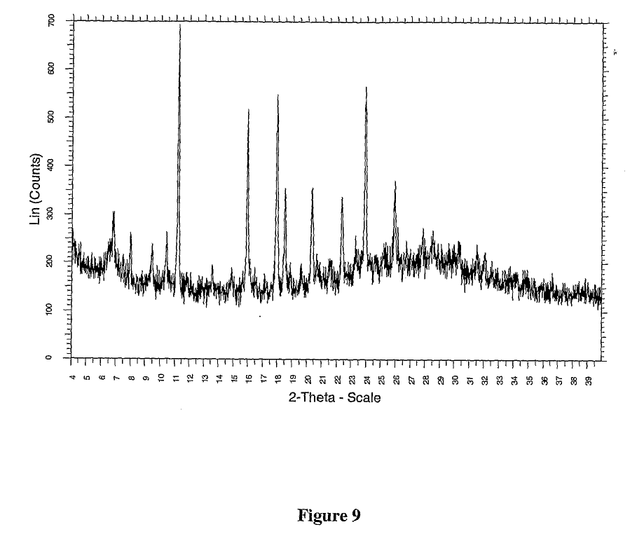

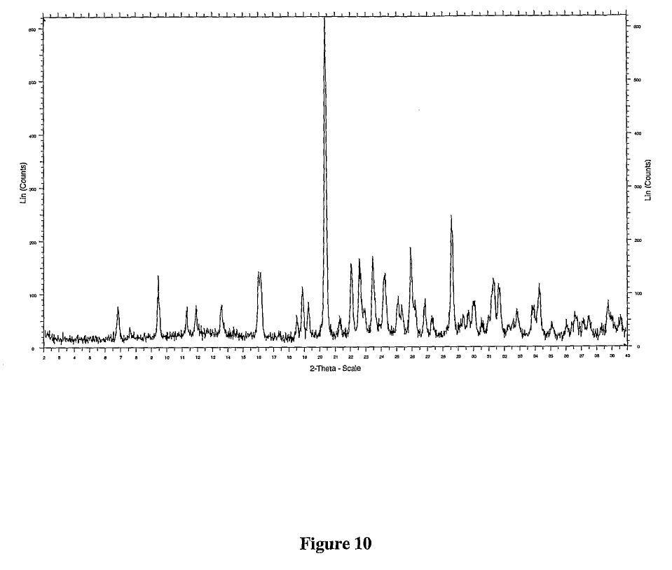

The slurry was prepared by adding 100 mg of Compound 1 tosylate to a 10 mL vial and to this was added 2 mL of the pH 6.5 buffer (prepared above), a magnetic stir bar was added and vial placed on a magnetic stirrer. Small aliquots of the suspension were removed after 15 minutes, 30 minutes, 45 minutes, 60 minutes and 120 minutes and each sample was placed on a metal plate on the D8 Diffractometer and an XRPD pattern determined immediately. The results after 0, 15 and 60 minutes are shown in FIGS. 7, 8 and 9, respectively. FIG. 10 shows the XRPD pattern for the free base and is provided for reference. These results show that there was little disproportionation of the salt to the free base under these conditions. Furthermore, no disproportionation of the tosylate salt of Compound 1 in the solid state was observed after 3 months storage at 40° C. and 5% relative humidity.

### Example 8

### In Vivo Investigation

### Study of Salts Versus Free Base in a Tablet Formulation

A dog study was performed to measure plasma levels of Compound 1 in fasted dogs following oral administration of 50 mg free base equivalent (fbe) doses in tablets of the free base and the tosylate salt (form 2 of the 1:1 stoichiometric salt), and 150 mg fbe as 3×50 mg tablets of the tosylate salt.

Tablets of Compound 1 were manufactured using a standard direct compression process. Three batches were made containing Compound 1 free base or Compound 1 tosylate as the active pharmaceutical ingredient (API). The generic formulation contained API (12.5% w/w), fast flo lactose (72.0% w/w), Avicel PH102 (10.0% w/w), AcDiSol (4.0% w/w), sodium lauryl sulfate (0.5% w/w) and magnesium stearate (1.0% w/w). The required quantity of each formulation component, excluding magnesium stearate, was weighed and charged to a mixing vessel. The powders were then mixed for 30 minutes using a tumble blender. The powder mix was then sieved through a 425 μm sieve before being mixed for a further 15 minutes using the tumble blender. The magnesium stearate was then added to the powder mix before blending manually for 20 seconds. The required quantity of powder mix for each tablet was weighed individually and charged to the die before being compressed manually using a hand-operated press. The Compound 1 free base tablets were compressed using 10 mm, round, plain, normal concave tooling at a compression force of approximately 0.1 tonnes (compaction pressure of approx. 10.8 MPa). The Compound 1 tosylate tablets were compressed using 12.5 mm, round, plain, normal concave tooling at a compression force of approximately 0.5 tonnes (compaction pressure of approx. 34.7 MPa). Lower compaction pressures were used for the Compound 1 free base as these formulations were observed to consolidate readily at higher pressures leading to prolonged dissolution times. In contrast, the Compound 1 tosylate formulation was observed to produce more robust tablets with acceptable dissolution profiles at higher compaction pressures, and was therefore less susceptible to variation in drug release with compaction pressure.

Single doses of 50 mg or 150 mg free base equivalent tablets were administered orally to six fasted Alderley Park Beagle Dogs weighing 11 to 15 kg and at least 9 months old on each of six dosing days. The doses were chosen as likely therapeutic doses. The tablet formulations were dosed orally followed by a 20 mL water wash to aid the passage of the tablet.

Dogs were fed about 400 g of Harlan Teklad 2021 each day and allowed water ad libitum. Whole blood (2 mL) in EDTA tubes were taken from the jugular vein immediately prior to dosing and at 0.5, 1, 2, 3, 4, 5, 6, 8, 12, 18, 24, 36 and 48 hours. The blood was centrifuged at 3000 rpm for 15 minutes and plasma removed into plain blood tubes and the plasma stored at −20° C. until analysis. Plasma (50 mcL) was analysed for Compound 1 concentration.

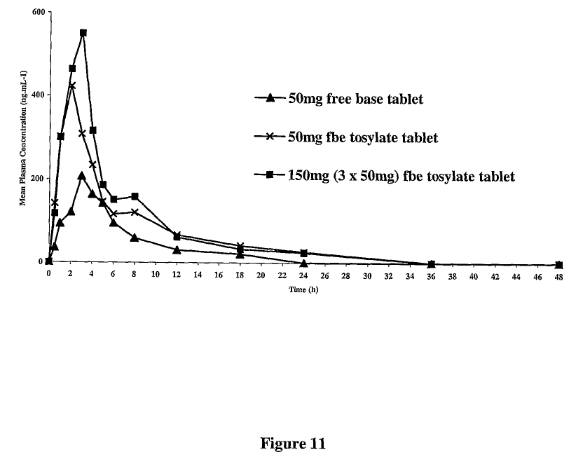

Mean plasma concentration profiles for Compound 1 seen after oral dosing are shown in FIG. 11, where the line represented by x represents the formulation which included Compound 1 tosylate salt at a dosage level of 50 mg free base equivalent, the line represented by ▪ represents the formulation which included Compound 1 tosylate salt at a dosage level of 150 mg free base equivalent, and the line represented by ▴ shows the results of a similar formulation comprising 50 mg Compound 1 present as the free base. It appeared that when Compound 1 was dosed as the tosylate salt, a substantial increase in exposure was produced.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A tosylate salt of Compound 1.

2. A tosylate salt of Compound 1 according to claim 1 in anhydrous form

3. A tosylate salt according to claim 1 or claim 2 wherein the ratio of Compound 1 to tosylate ion present is 1:1.

4. A tosylate salt according to claim 1 or claim 2 wherein the ratio of Compound 1 to tosylate ion present is 2:1.

5. A polymorph of the tosylate salt of Compound 1 according to claim 3.

6. A tosylate salt of Compound 1 according to any one of the previous claims which is crystalline.

7. Crystalline Form 1 of a tosylate salt of Compound 1.

8. Crystalline Form 2 of a tosylate salt of Compound 1.

9. A tosylate salt of Compound 1, wherein said tosylate salt of Compound 1 has an X-ray powder diffraction pattern with at least one specific peak at about 18.43°.

10. A tosylate salt of Compound 1 according to claim 9, wherein said tosylate salt of Compound 1 has an X-ray powder diffraction pattern with specific peaks at about 2-theta equal to 18.43°, 23.85°, 17.86°, 25.91°, 11.08°, 27.78° and 22.30°.

11. A tosylate salt of Compound 1 according to claim 1 which has a powder X-ray diffraction pattern substantially the same as the X-ray powder diffraction pattern shown in FIG. 2.

12. A tosylate salt of Compound 1 which has a powder X-ray diffraction pattern substantially the same as the X-ray powder diffraction pattern shown in FIG. 1 or FIG.

13. A method for preparing a tosylate salt of Compound 1 according to any one of claims 1 to 12, said method comprising:
(i) reacting a slurry of Compound 1 in an organic liquid with toluene sulfonic acid; and
(ii) precipitating the salt from the resultant solution.

14. A method according to claim 13 wherein the mole ratio of the amount of Compound 1: toluene sulfonic acid is in the range of from 0.95:1 to 1.05:1.

15. A method according to claim 13 or claim 14 wherein the organic liquid is a C₁₋₆ alcohol.

16. A pharmaceutical composition which comprises a tosylate salt of Compound 1 as defined in any one of claims 1 to 12 in association with a pharmaceutically acceptable excipient or carrier.

17. A tosylate salt of Compound 1 according to any one of claims 1 to 12 for use as a medicament for the treatment of disease states mediated through MEK.

18. The use of a tosylate salt of Compound 1 according to any one of claims 1 to 12 in the manufacture of a medicament for use in the treatment of disease states mediated through MEK.

19. A method for treating disease states mediated through the MEK, in a warm-blooded mammalian need of such treatment which comprises administering to said mammal an effective amount of a tosylate salt of Compound 1 according to any one of claims 1 to 12, or a pharmaceutical composition according to claim 16.
